(19) United States
(12) Patent Application Publication
(10) Pub. No.: US 2021/0023115 A1
(43) Pub. Date: Jan. 28, 2021

(54) COMPOSITION OF ANTI-TUMOR COMPOUND DRUG AND APPLICATION THEREOF IN FIGHTING TUMORS

(71) Applicant: HANGZHOU XINGAO BIOTECHNOLOGY CO. LTD, Hangzhou (CN)

(72) Inventors: Xiangshi TAN, Hangzhou (CN); Jianwu PEI, Hangzhou (CN); Yueru ZHANG, Hangzhou (CN); Xiang WANG, Hangzhou (CN); Hao CHENG, Hangzhou (CN)

(73) Assignee: HANGZHOU XINGAO BIOTECHNOLOGY CO. LTD

(21) Appl. No.: 17/066,496

(22) Filed: Oct. 9, 2020

Related U.S. Application Data
(63) Continuation of application No. PCT/CN2019/088456, filed on May 25, 2019.

(30) Foreign Application Priority Data
Jun. 9, 2018 (CN) 201810590933.9

(51) Int. Cl. A61K 31/7084; A61K 9/127

(52) U.S. Cl. CPC A61K 31/7084; A61K 9/127

(57) ABSTRACT

The present invention belongs to the technical field of biomedicine, and discloses a multi-functional anti-tumor compound drug. A composition of the anti-tumor compound drug comprises an agonist of an innate immune pathway (STING) and an inhibitor of phosphodiesterase ENPP1. Both STING and ENPP1 are located on the endoplasmic reticulum membrane. An activator and the agonist of STING are hydrolysis substrates of ENPP1. The compound drug can have two-pronged effects. The compound anti-tumor drug has a better anti-tumor effect than that by using an innate immune agonist alone. Therefore, the innovative compound anti-tumor drug has an efficient clinical application prospect.

### TECHNICAL FIELD

The present invention belongs to the technical field of biomedicine, and particularly relates to a composition of an anti-tumor compound drug and an application thereof in fighting tumors.

### BACKGROUND

As one of the major diseases that seriously endanger human life and health, tumor is characterized by excessive cell proliferation and abnormal differentiation. WHO experts predict that by 2020, the incidence of tumors in global population will reach 20 million, and the death toll will reach 12 million. Tumors will become the number one killer of mankind in this century and pose the most serious threat to human survival. The incidence rate and mortality rate of lung cancer, colorectal cancer, gastric cancer, liver cancer, etc. are among the forefront of various malignant tumors. According to statistics in 2012 Chinese Cancer Registry Annual Report released by the National Central Cancer Registry of China, about 3.12 million new cancer cases occur each year, with an average of 8550 people per day and 6 people per minute being diagnosed with cancer across the country. In terms of disease entities, lung cancer, gastric cancer, colorectal cancer, liver cancer and esophageal cancer rank among the top five malignant tumors in the country. As the incidence rate and mortality rate of malignant tumors increase year by year, the demand for malignant tumor treatment is increasing.

As a secondary messenger molecule, cyclic dinucleotide cGAMP induces the production of interferon IFN-β and other cytokines by activating the STING protein pathway on the endoplasmic reticulum membrane, regulates the expression of downstream protein, and induces cell growth arrest and apoptosis. The STING pathway can regulate the innate immune recognition of immunogenic tumors and promote the anti-tumor effect of the interferon. IFN-γ plays an anti-tumor effect in vivo through TRAIL (tumor necrosis factor-related apoptosis-inducing ligand), and promotes the apoptosis of tumor cells. cGAMP is a key irritant of innate immune response and an endogenous activator of STING. Therefore, cGAMP has an immune anti-tumor effect.

STING is a transmembrane protein of the endoplasmic reticulum which has a phosphodiesterase (hydrolase) of ENPP1. ENPP1 hydrolase can degrade 2′3′-cGAMP, and has a very wide substrate specificity, including ATP and NAD+. Experiments show that 2′3′-cGAMP is a good substrate for recombinant ENPP1. Therefore, effectively inhibiting the catalytic activity of ENPP1 can inhibit the hydrolysis of the STING activator by ENPP1 hydrolase, prolong the metabolic cycle thereof and improve the effect of the drug.

Based on the above principles, the present invention discloses an anti-tumor compound drug which comprises an activator of an innate immune pathway (STING) and an inhibitor of phosphodiesterase ENPP1, has two-pronged effects, and achieves an improved immune anti-tumor effect. Therefore, the immune anti-tumor compound drug has a good clinical application prospect.

### SUMMARY

The purpose of the present invention is to provide a multi-functional anti-tumor compound drug which comprises an activator 2′3′-cGAMP (or an agonist of derivatives thereof) of an innate immune pathway (STING) and an inhibitor of phosphodiesterase ENPP1, has two-pronged effects, and achieves an improved immune anti-tumor effect. Therefore, the immune anti-tumor compound drug has a good clinical application prospect.

The cyclic dinucleotide cGAMP of the present invention refers to 2′3′-cGAMP or Cyclic [G(2′,5′)pA(3′,5′)p] unless otherwise specified.

### DETAILED DESCRIPTION

The present invention is described in detail below through embodiments. In the present invention, the following embodiments are only used for preferably illustrating the present invention, not used for limiting the scope of the present invention.

### Embodiment 1: Preparation of Cyclic Dinucleotide cGAMP and Derivative Thereof

cGAMP or a derivative thereof (cyclic GMP-AMP) is synthesized by cyclic cGMP-AMP dinucleotide synthase (cGAS) through catalysis under the activation conditions after binding DNA according to literature methods. (Li P. W, et al., Immunity, 2013, 39(6), 1019-1031.)

### Embodiment 2: Preparation of Inhibitor of ENPP1

ATP and derivatives thereof are hydrolysis substrates of ENPP1, and modified analogs of ATP can selectively inhibit the enzymatic catalytic activity of ENPP1. The inhibitor of ENPP1 of the present invention and a preparation route thereof are shown in FIG. 1.

### Embodiment 3: Preparation of Innovative Anti-Tumor Compound Drug-Targeted Liposomes

(1) Raw materials of liposomes: lecithin (lipoid EPCs), cholesterol (CH), polyethylene glycol, etc. are all purchased from Sigma company. (2) Folate-targeted liposomes are prepared according to literature methods. (Chen X., et al., Int J Nanomedicine, 2012, 7:1139-1148; Waldrep J. C., et al., Int J Pharm, 1998, 160(2):239-249).

(3) Encapsulation of anti-tumor compound drug by folate-targeted liposomes

A 120 mmol/L ammonium sulfate solution is added to a phospholipid membrane and shaken (120 rpm, 5 minutes) to form a blank liposome solution. The blank liposome solution is dialyzed in ultrapure water overnight. The compound drug is dissolved in the ultrapure water, added to the blank liposome solution, and incubated at 65° C. for 20 minutes. Particle diameters are reduced by ultrasonic in a water bath, and unencapsulated drug is removed by ultrafiltration using an ultrafiltration tube (MWCO=3000 Da).

(4) Characterization of anti-tumor compound drug-targeted liposomes

(a) Characterization of particle diameters

The particle diameters and Particle Diameter Distribution (PDI) of liposomes are measured by Dynamic Light Scattering (DLS). The basic principle is that tiny particles suspending in liquid will move randomly (Brownian motion). When light passes through a colloid, the particles will scatter the light, and a light signal can be detected at a certain angle. Large particles move slowly, and the intensity of the scattered light spots will also fluctuate slowly; small particles move rapidly, and the density of the scattered light spots will also fluctuate rapidly. Finally, the particle diameters and particle diameter distribution are calculated by light intensity fluctuation change and a light intensity correlation function. PDI indicates the uniformity of particle diameters and is a concept of variance. The prepared liposomes have a particle diameter of about 70 nm.

(b) Zeta potential

Zeta potential is the potential difference between a continuous phase and a fluid stabilization layer attached to dispersed particles, and is generally used to evaluate or predict the physical stability of a particle dispersion system. Generally, the higher the absolute value of the Zeta potential is, the greater the electrostatic repulsion between particles will be, and the better the physical stability will be. Generally, if the absolute value of the Zeta potential reaches 30 mV, the system is considered to be relatively stable. The absolute value of the Zeta potential of the liposomes prepared by the present invention is 29 mV, so the stability is good.

### Embodiment 4: Test of Anti-Tumor Effect (i.e., The Inhibition Effect on the Growth of Subcutaneous Transplanted Tumors in Animals) of the Anti-Tumor Compound Drug with a Tumor-Bearing Mouse Model

Animals

Genus, strain, sex, weight, source, certificate

BALB/c ordinary mice and C57/BL6 ordinary mice, male, 18-20 g in weight, 7-8 weeks old, SPF grade, purchased from Shanghai SLAC Laboratory Animal Co., Ltd. [Laboratory Animal Quality Certificate Number: SCXK (Shanghai) 2007-0005].

Feeding conditions

All mice are free to forage and drink, and are fed at room temperature of (23±2)° C. Feed and water are sterilized at high pressure, and all experimental feeding processes are SPF grade.

Dose setting

Mice are given intravenous injections, and one dose group is set: cGAMP, 10 mg/kg; compound anti-tumor drug, 10 mg/kg

Test controls

Negative control: normal saline solution

Positive control: cGAMP, 10 mg/kg in dose

Administration method

Administration route: administration by intraperitoneal injection

Administration volume: 100 μl/animal

Number of times of administration: continuous administration for 21 days, once every day

Number of animals in each group: 10

Tumor cell lines

Mouse colorectal cancer cell line CT26, mouse Lewis lung cancer cell line LL/2, human ovarian cancer cell line SK-OV-3, human melanoma cell line A375, and human gastric cancer cell line MNK-45, which are all purchased from the Cell Bank of the Chinese Academy of Sciences.

Main test steps

1. Establishment and intervention of mouse tumor model

Cells are cultured and passaged, are collected in the logarithmic phase of cells, and are made into cell suspension with a concentration of (1.0×10⁷) per milliliter; 0.2 ml of the cell suspension is injected into the axilla of the right forelimb of each mouse (the number of cells is 2.0×10⁶/mouse), after about 10 days, when growing to about 5 mm in diameter, the tumors are successfully induced, and are randomly divided into 4 groups, including: A: negative control group; B: cGAMP group; C: anti-tumor compound drug group; and D: anti-tumor compound drug-targeted liposome group. The administration is continued for 21 days. 21 days later, the mice are killed and the tumors are weighed. Tumor inhibition rate=[1−mean tumor weight of experimental group/mean tumor weight of group A)]×100%.

2. Statistical analysis

Data is expressed with x±s, and processed with SPSS10.0 software. The significance of the difference between tumor weights of all groups is compared by a one-way ANOVA test. The significance level a=0.05.

Results

After subcutaneous inoculation of tumor cells in mice, a subcutaneous transplanted tumor model is successfully prepared. The innovative anti-tumor compound drug, the targeted liposomes thereof, and the activator (cGAMP) of an innate immune pathway alone can obviously inhibit the growth of tumors. After administration for 21 days, the weights of tumors are all significantly lower than those of the negative control group (P<0.05, P<0.01), indicating that the anti-tumor compound drug has a better anti-tumor effect. The specific results are shown in Tables 1-5.

Table 1 Effect of anti-tumor compound drug on subcutaneous transplanted tumor of BalB/C mouse colorectal cancer cell CT26

(n=10, mean±SD)

Note: *P<0.05 vs negative control group; **P<0.01 vs negative control group.

Table 2 Effect of anti-tumor compound drug on subcutaneous transplanted tumor of C57 mouse Lewis lung cancer cell line LL-2 (n=10, mean±SD)

Note: *P<0.05 vs negative control group; **P<0.01 vs negative control group.

Table 3 Effect of anti-tumor compound drug on mouse subcutaneous transplanted tumor of human melanoma cell line A375 (n=10, mean±SD)

Note: *P<0.05 vs negative control group; **P<0.01 vs negative control group.

Table 4 Effect of anti-tumor compound drug on mouse subcutaneous transplanted tumor of human gastric cancer cell line MNK-45 (n=10, mean+SD)

Note: *P<0.05 vs negative control group; **P<0.01 vs negative control group.

Table 5 Effect of anti-tumor compound drug on mouse subcutaneous transplanted tumor of human ovarian cancer cell line SK-OV-3 (n=10, mean±SD)

Note: *P<0.05 vs negative control group; **P<0.01 vs negative control group.

Study on acute toxicity of anti-tumor compound drug of embodiment 4

Experimental material

20 ICR mice (purchased from Shanghai SLAC Laboratory Animal Co., Ltd. [Laboratory Animal Quality Certificate Number: SCXK (Shanghai) 2007-0005]), half male and half female, 20-25 g in weight. The animals are fed with pellet feed and are free to forage and drink.

The anti-tumor compound drug is prepared according to embodiment 2, and is made into a solution with a concentration of 200 mg/mL by using normal saline.

Experiment method

Each ICR mouse is given a single intraperitoneal injection of 2 g/kg of a sustained-release preparation of the compound immune anti-tumor drug according to the weight of the mouse, and the toxic reaction and death of the mice within 14 days after administration are observed. Results show that after administration by a single tail intravenous injection, the mice move normally. No death of the mice occurs within 14 days after the administration. On the 15^{th} day, all mice are killed and dissected, various organs are visually inspected, and no obvious pathological change is seen.

Experimental results

The above-mentioned acute toxicity experiment results show that the maximum tolerated dose MTD for administration by intravenous injection is not less than 2 g/kg, indicating that the acute toxicity of the compound immune anti-tumor drug is low.

### DESCRIPTION OF DRAWINGS

FIG. 1. 8-aza-ATP-α-methylene-γ-sulfhydryl

FIG. 2. Inhibitor compound of phosphodiesterase ENPP1

1. A composition of an anti-tumor compound drug, comprising an agonist (activator) of an innate immune pathway (STING pathway) and an inhibitor of phosphodiesterase ENPP1, wherein the agonist (activator) of the innate immune pathway (STING pathway) includes but is not limited to cyclic dinucleotide cGAMP and various derivatives and analogs thereof (a composition of cyclic dinucleotide comprises two ATPs, two GTPs, or mixed ATP/GTP and substituted derivatives or modified analogs of ATP/GTP), and the inhibitor of phosphodiesterase ENPP1 includes but is not limited to ATP and various derivatives and analogs of ATP, or a small molecule inhibitor of ENPP1 obtained through high-throughput screening, etc.

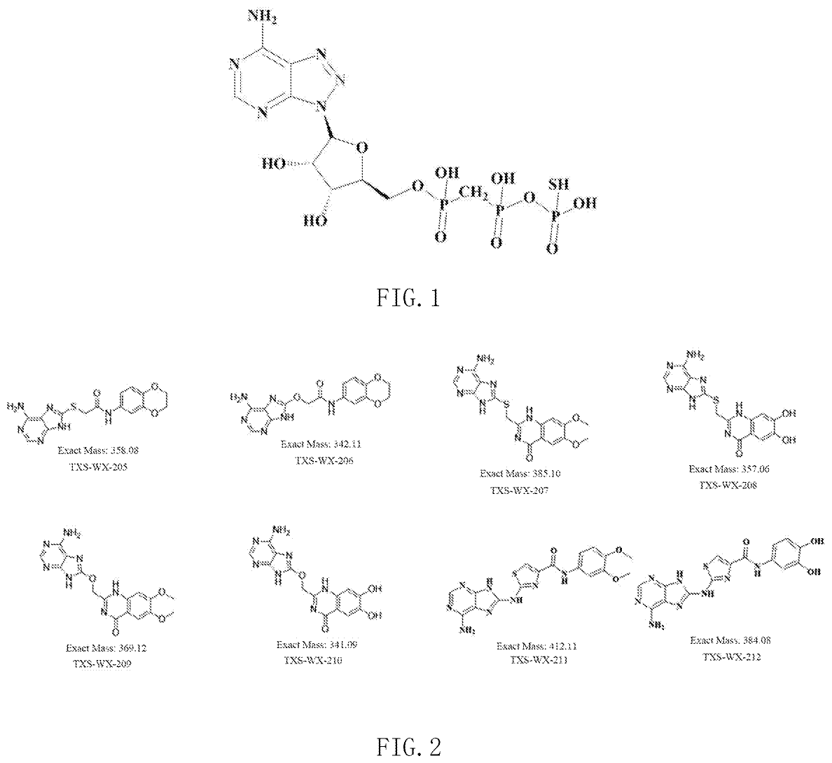

2. The novel inhibitor of phosphodiesterase ENPP1 includes but is not limited to compounds (chemical structures and preparation methods) listed in FIGS. 1-2.

3. An application of the compounds of the compound anti-tumor drug in fighting tumors and an application thereof in preparing anti-tumor drugs.

4. Liposomes and targeted liposomes that can be prepared from the anti-tumor compound drug according to claim 1 (including but not limited to folate-targeted liposomes and immune-targeted liposomes).

5. The anti-tumor compound drug according to claim 1, wherein the anti-tumor compound drug is applied to treatment of various tumors, including but not limited to colorectal cancer, ovarian cancer, prostate cancer, testicle cancer, lung cancer, nasopharyngeal cancer, esophagus cancer, malignant lymphoma, head and neck cancer, thyroid cancer, osteogenic sarcoma and various other solid tumors.

6. The anti-tumor compound drug according to claim 1, including but not limited to an anti-tumor drug, wherein the compound drug can be applied to other indications based on STING innate immune pathway activation/enhanced treatment, including but not limited to: neurodegenerative diseases (AD, PD), cardiovascular and cerebrovascular diseases, diabetes, rheumatoid arthritis, multiple sclerosis and other related diseases.
